(19) United States
(12) Patent Application Publication
(10) Pub. No.: US 2023/0404908 A1
(43) Pub. Date: Dec. 21, 2023

(54) Compositions And Methods For Treating With A Combination Of Alternating Electric Fields And Trastuzumab

(71) Applicant: NOVOCURE GMBH, Root D4 (CH)

(72) Inventor: EunHo Kim, Haifa (IL)

(21) Appl. No.: 17/828,794

(22) Filed: May 31, 2022

(51) Int. Cl. A61K 9/00; C07K 16/32; A61K 39/395; A61K 49/00; A61P 35/00; A61N 1/32

(52) U.S. Cl. CPC A61K 9/0009; C07K 16/32; A61K 39/39558; A61K 2039/505; A61P 35/00; A61N 1/327; A61K 49/0058

(57) ABSTRACT

Disclosed are methods of treating a subject in need thereof comprising applying an alternating electric field, at a frequency for a period of time, to a target site of the subject in need thereof, and administering trastuzumab to the target site of the subject in need thereof. Disclosed are methods of increasing accumulation of trastuzumab at a target site of a subject in need thereof comprising applying an alternating electric field, at a frequency for a period of time, to the target site of the subject in need thereof; and administering trastuzumab to the target site of the subject in need thereof, wherein the accumulation of the trastuzumab increases at the target site compared to administering the trastuzumab at the target site without applying an alternating electric field. Disclosed are methods of inhibiting cancer cell proliferation or increasing apoptosis of cancer cells at a target site of a subject in need thereof comprising applying an alternating electric field, at a frequency for a period of time, to the target site of the subject in need thereof; and administering trastuzumab to the target site of the subject in need thereof, wherein cancer cell proliferation is inhibited or wherein apoptosis of cancer cells is increased.

### BACKGROUND

One of the reasons for the increased mortality rate among U.S. women is the ever-increasing breast cancer cases. According to the American Cancer Society's 2015 census, around 60,290 breast carcinoma cases (in situ) were estimated to be identified. Further, of all the malignant tumors, breast cancer makes up for 7-10%, with around 3-4% yearly increase in China. It has also been found that increased activation of human epidermal growth factor receptor 2 (HER2) tyrosine kinase receptor gene is a negative prognostic factor that is responsible for early node-positive breast cancer. Hence, an enhanced explanation of HER2 gene's mechanism and action could lead to the reshaping of the classification, prognosis, as well as treatment of the disease. Further, one finds a 15-20% highly expressed HER2 gene in various breast cancers which tends to modulate various cell processes including cell survival, proliferation, angiogenesis, invasion and metastasis. Recently, a humanized monoclonal anti-ERBB2 antibody known as Trastuzumab (Herceptin) has been discovered which significantly improves clinical outcome for early and advanced HER2-positive breast cancer. Trastuzumab affects HER2 by inhibiting its dimerization and hence affecting the growth signals which eventually shuts down the HER 2 receptor gene's expression. This drug also targets the phosphatidylinositol 3-kinase (PI3K)/AKT/mTOR pathway and RAS/RAF/MEK/MAP kinase (MAPK) pathways. Besides this, the Fc portion of trastuzumab participates in antibody-dependent cellular cytotoxicity (ADCC) function. Though the antibody showed a high rate of initial effectiveness, the patients suffering from metastatic breast cancer stage developed a primary resistance against the antibody. Therefore, this treatment failed to prove effective in this case. Thus, this drawback poses a greater challenge in the treatment of HER2-positive breast cancer patients.

Tumor Treating Fields, better abbreviated as TTFields or TTF, is the therapy comprising of alternating electromagnetic field employed with low-intensity electrical fields to suppress cancer cell proliferation in the body. This therapy targets to induce an electric field inside the human body to suppress the proliferation and invasion of cancerous cells. This treatment induces apoptosis and hence shows an ability to annihilate the cancerous cells. FDA has granted its approval for the application of TTFields to treat patients with recurrent glioblastoma multiforme, or GBM. This technique is found to increase the life expectancy of the patients. In the case of newly diagnosed GBM, this technique can be coupled with temozolomide preceding the surgery (www.accessdata.fda.gov/scripts/cdrh/cfdocs/cfpma/pma.cfm?id=P100034) (www.accessdata.fda.gov/scripts/cdrh/cfdocs/cfpma/pma.cfm?id=P100034S013). The American Society of Clinical Oncology (ASCO) regards this treatment as an advancement in cancer treatment due to its novel approach, effectiveness, and low toxicity profile. The National Comprehensive Cancer Network (NCCN), recommends TTFields as a category 2A treatment for patients with newly diagnosed GBM and resulting in a good performance status (www.nccn.org/professionals/physician_gls/f_guidelines. asp). In contrast to the typically employed methods (chemotherapy and radiation), TTF does not cause side effects like pain, nausea, fatigue or diarrhea. Still, this technique is associated with some flaws noted in the trials which include topical skin rashes caused by prolonged electrode use. However, the effectiveness of TTF is considered to be similar to that of chemotherapy or radiotherapy. Subsequently, TTF's and trastuzumab's combined antitumor effects on HER2-positive breast cancer representative cell lines alongside a tumor xenograft model was studied. The results showed that the TTF therapy can enhance significantly the growth inhibition induce by trastuzumab.

### BRIEF SUMMARY

Disclosed are methods of treating a subject in need thereof comprising applying an alternating electric field, at a frequency for a period of time, to a target site of the subject in need thereof; and administering trastuzumab to the target site of the subject in need thereof

Disclosed are methods of increasing accumulation of trastuzumabat a target site of a subject in need thereof comprising applying an alternating electric field, at a frequency for a period of time, to the target site of the subject in need thereof; and administering trastuzumabto the target site of the subject in need thereof, wherein the accumulation of trastuzumabincreases at the target site compared to administering trastuzumab at the target site without applying an alternating electric field.

Disclosed are methods of inhibiting cancer cell proliferation at a target site of a subject in need thereof comprising applying an alternating electric field, at a frequency for a period of time, to the target site of the subject in need thereof; and administering trastuzumab to the target site of the subject in need thereof, wherein cancer cell proliferation is inhibited.

Disclosed are methods of increasing apoptosis of cancer cells at a target site of a subject in need thereof comprising applying an alternating electric field, at a frequency for a period of time, to the target site of the subject in need thereof; and administering trastuzumabto the target site of the subject in need thereof, wherein apoptosis of cancer cells is increased.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1B depict the effect of trastuzumab-treatment on the viability of HER2-positive cell. FIG. 1A: TRZ inhibited HER2-positive cell viability in a dose-dependent manner. FIG. 1B: Cell viability was evaluated using MTT assay for Jimt1 and BT474 cells treated with the specified amounts of TRZ; *p<0.05.

FIGS. 2A-2C depict effect of trastuzumab-treatment with TTF on the viability of HER2-positive cell. FIG. 2A: Jimt1 cells were treated in dimethyl sulfoxide (DMSO, vehicle), TRZ (5 μg/ml), TTF (1.5 V/cm), combination for 48 hours and the morphological changes were monitored under phase-contrast microscopy (Magnification ×400). FIG. 2B: The cell viability was ascertained by trypan blue exclusion assay. FIG. 2CA: Colony formation assays were carried out using Jimt1 cells treated with the specified treatment for 7-9 days (n=3); *p<0.05, **p<0.01, ***p<0.001.

FIGS. 3A-3C depict anti-trastuzumab-resistant tumor effects of TTF in a BT474-cell line human breast cancer xenograft model. FIG. 3A: Nude mice bearing BT474R cells as xenografts were treated using control (saline; “CTL”), trastuzumab (“TRZ”), TTF, or combination of trastuzumab and TTF (“TTF+TRZ”). FIG. 3B: Treatment effects on tumor volume. FIG. 3C: Representative hematoxylin and eosin (H & E) staining of Ki-67. Data are presented as the mean±standard error of the mean (n=5), *P<0.05.

FIGS. 4A-4C depict that combination treatment induces cell apoptosis in trastuzumab-resistant HER2-positive cell line. FIG. 4A: Jimt1 cells were treated with DMSO, TRZ (5 μg/ml), TTF, or combination for 48 hours. Immunoblotting (or western blotting) was performed on cell lysates (30 μg) with antibodies against pHER2, HER2, Cleaved PARP, Bcl-2, and β-actin. FIG. 4B: Representative of TUNEL. Data are presented as per the mean±standard error of the mean (n=10), *P<0.05. FIG. 4C: TUNEL-positive cells were stained and imaged with 10× objective lenses using In cell analyzer. Apoptotic cells are shown in light grey.

FIG. 5 depicts effects of TTF on the growth factor receptor 2 of human epidermal and downstream signaling pathways. pAKT, pERK, and pHER2 expressions in xenografts were examined using immunohistochemistry. *p<0.05, **p<0.01.

FIG. 6 depicts that TTF increases the penetration of trastuzumab. Fluorescent images were obtained with 10× objective lenses, using In cell analyzer. The channels are as follow: DAPI for nuclei (shown in blue), Alexa 488-TRZ (shown in green) and Rhodamine lectin to detect blood vessel (shown in red). The penetration of TRZ from the tumor vessel was plotted with line profiling by intensity measurement from the vessel in various ROI from peripheral and central regions in each tumor. Additionally, the area under the curve was calculated from three tumors with 10 sections in each tumor in two groups. *p<0.05.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “an antibody” includes a plurality of such antibodies, reference to “the cell” is a reference to one or more cells and equivalents thereof known to those skilled in the art, and so forth.

As used herein, a “target site” is a specific site or location within or present on a subject or patient. For example, a “target site” can refer to, but is not limited to a cell (e.g. a cancer cell), population of cells, organ, tissue, or a tumor. Thus, the phrase “target cell” can be used to refer to target site, wherein the target site is a cell. In some aspects, a “target cell” can be a cancer cell. In some aspects, organs that can be target sites include, but are not limited to, abdominal organs (e.g. stomach, intestine) or breast,. In some aspects, a cell or population of cells that can be target site or target cell include, but are not limited to, breast tissue cells or abdominal cells. In some aspects, a “target site” can be a tumor target site.

A “tumor target site” is a site or location within or present on a subject or patient that comprises or is adjacent to one or more cancer cells, previously comprised one or more tumor cells, or is suspected of comprising one or more tumor cells. For example, a tumor target site can refer to a site or location within or present on a subject or patient that is prone to metastases. Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g. a target site such as a cell). In some aspects, the alternating electrical field can be in a single direction or multiple directional. In some aspects, alternating electric fields can be delivered through two pairs of transducer arrays that generate perpendicular fields within the target site. For example, for the Optune™ system (an alternating electric fields delivery system) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

As used herein, an “alternating electric field” applied to a tumor target site can be referred to as a “tumor treating field” or “TTField.” TTFields have been established as an anti-mitotic cancer treatment modality because they interfere with proper micro-tubule assembly during metaphase and eventually destroy the cells during telophase, cytokinesis, or subsequent interphase. TTFields target solid tumors and is described in U.S. Pat. No. 7,565,205, which is incorporated herein by reference in its entirety for its teaching of TTFields

In-vivo and in-vitro studies show that the efficacy of TTFields therapy increases as the intensity of the electrical field increases. Therefore, optimizing array placement on a subject to increase the intensity in the target site or target cell is standard practice for the Optune system. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target site or target cell as a function of the positions of the array and, in some aspects, take account for variations in the electrical property distributions within the heads of different patients.

The term “subject” refers to the target of administration, e.g. an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field. For example, the subject of administration can be a subject with early and advanced HER2-positive breast cancer.

By “treat” is meant to administer or apply a therapeutic, such as alternating electric fields and a vector, to a subject, such as a human or other mammal (for example, an animal model), that has breast or stomach cancer or has an increased susceptibility for developing breast or stomach cancer, in order to prevent or delay a worsening of the effects of the disease or infection, or to partially or fully reverse the effects of breast or stomach cancer. For example, treating a subject having breast or stomach cancer can comprise delivering a therapeutic to a cell in the subject.

By “prevent” is meant to minimize or decrease the chance that a subject develops breast or stomach cancer.

As used herein, the terms “administering” and “administration” refer to any method of providing trastuzumab to a target site or subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat breast or stomach cancer. In further various aspects, a preparation can be administered prophylactically; that is, administered for prevention of breast or stomach cancer. In an aspect, the skilled person can determine an efficacious dose, an efficacious schedule, or an efficacious route of administration so as to treat a subject. In some aspects, administering comprises exposing or applying. Thus, in some aspects, exposing a target site or subject to alternating electrical fields or applying alternating electrical fields to a target site or subject means administering alternating electrical fields to the target site or subject.

As used herein, a “therapeutically effective amount” is an amount of a composition, or trastuzumab, that provides a therapeutic benefit to an individual or subject. For example, a therapeutically effective amount of trastuzumab is an amount that treats, alleviates, ameliorates, relieves, alleviates symptoms of, prevents, delays onset of, inhibits progression of, reduces severity of, and/or reduces incidence of breast or stomach cancer. In one embodiment, a therapeutically effective amount of trastuzumab will result in an improvement to, or prevents or slows the worsening of, one or more indicators or symptoms of breast or stomach cancer, such as those described herein. As used herein, “treating” a subject with breast or stomach cancer includes administering a therapeutically effective amount of trastuzumab.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise alternating electric fields. In some aspects, the alternating electric field used in the methods disclosed herein is a tumor-treating field. In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 kHz and 1 MHz. In some aspects, the frequency of the alternating electric field is between 100 and 500 kHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 180 and 220 kHz, or between 210 and 400 kHz. In some aspects, the frequency of the alternating electric fields can be electric fields at 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be around 300 kHz.

In some aspects, the field strength of the alternating electric fields can be between 0.5 and 4 V/cm RMS. In some aspects, the field strength of the alternating electric fields can be between 1 and 4 V/cm RMS. In some aspects, different field strengths can be used (e.g., between 0.1 and 10 V/cm). In some aspects, the field strength can be 1.75 V/cm RMS. In some embodiments the field strength is at least 1 V/cm RMS. In some aspects, the field strength can be at least 0.9 V/cm RMS. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the exposure may last for at least 6 hours, at least 12 hours, at least 24 hours, at least 36 hours, at least 48 hours, or at least 72 hours or more.

The disclosed methods comprising applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell.

### C. Trastuzumab

Disclosed are trastuzumab antibodies to be administered to a target site of a subject in need thereof. In some aspects, trastuzumab is administered as a combination therapy or treatment with alternating electric fields.

Disclosed are compositions comprising one or more trastuzumab antibodies.

In some aspects, the composition can be a pharmaceutical composition (e.g., formulation, preparation, medicament) comprising, or consisting essentially of, or consisting of as an active ingredient, trastuzumab as described herein, and a pharmaceutically acceptable carrier, diluent, or excipient.

Disclosed are compositions and formulations of trastuzumab with a pharmaceutically acceptable carrier or diluent. For example, disclosed are pharmaceutical compositions, comprising trastuzumab, and a pharmaceutically acceptable carrier.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. In the methods described herein, delivery of the disclosed compositions to cells can be via a variety of mechanisms. The pharmaceutical composition may be administered in a number of ways depending on whether local or systemic treatment is desired, and on the area to be treated.

1. Delivery of Compositions

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

### D. Methods of Treating

Disclosed are methods of treating a subject in need thereof comprising applying an alternating electric field, at a frequency for a period of time, to a target site of the subject in need thereof; and administering trastuzumab to the target site of the subject in need thereof

In some aspects, the target site comprises a cell. For example, the cell can be, but is not limited to a cancer cell . In some aspects, a cancer cell can be, but is not limited to, a breast cancer cell or a stomach cancer cell,

In some aspects, the alternating electric field is applied before, after, or simultaneously with administering the trastuzumab. In some aspects, the trastuzumab is administered prior to, simultaneous with or after applying the alternating electric field Applying the alternating electric field before administering the trastuzumab can include seconds, minutes, or hours before administering the trastuzumab. Applying the alternating electric field after administering the trastuzumab can include seconds, minutes, hours, or days after administering the trastuzumab. Applying the alternating electric field simultaneously with administering the trastuzumab can include seconds or minutes before or after administering the trastuzumab. In some aspects, simultaneously applying the alternating electric field and the trastuzumab can include administering the trastuzumab while the alternating electric field is being applied. In some aspects, the step of applying the alternating electric field begins at least one hour before the given time.

In some aspects, the trastuzumab comprises a detectable agent. As used herein, a detectable agent, or label, is any molecule that can be associated with trastuzumab, directly or indirectly, and which results in a measurable, detectable signal, either directly or indirectly. Many such labels for conjugating or coupling to an antibody are known to those of skill in the art. Examples of detection agents can be, but are not limited to, radioactive isotopes, fluorescent molecules (fluorophore), phosphorescent molecules, enzymes, antibodies, and ligands.

In some instances, a label can be, but is not limited to, an isotope marker, colorimetric biosensors, or fluorescent labels. For example, fluorescent markers can be, but are not limited to, green fluorescent protein (GFP) or rhodamine fluorescent protein (RFP). Other labels can include biotin, streptavidin, horseradish peroxidase, or luciferase. Other examples of suitable fluorescent labels include, but are not limited to, fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, OR and Research Organics, Cleveland, Ohio.

In some aspects, the frequency of the alternating electric field is any of those described herein. In some aspects, the frequency of the alternating electric field is between 100 and 500 kHz. For example, in some aspects, the frequency of the alternating electric field is 150 kHz.

In some aspects, the alternating electric field has a field strength of any of those described herein. In some aspects, the alternating electric field has a field strength of between and 4 V/cm RMS. For example, in some aspects, the alternating electric field has a field strength of 0.9 V/cm RMS.

In some aspects, trastuzumab is administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g. computed tomography-guided, during surgery or biopsy) or via inhalation. In some aspects, the trastuzumab is administered in a pharmaceutical composition. For example, the pharmaceutical composition can be any of those described herein.

In some aspects, the subject in need thereof has cancer. In some aspects, the cancer can be breast or stomach cancer. For example, the cancer can be a HER2 positive cancer.

In some aspects, one or more of pAKT, pERK, and pHER2 expression is reduced as a result of treatment with alternating electric fields and trastuzumab.

Disclosed are methods of treating a subject in need thereof comprising applying an alternating electric field, at a frequency for a period of time, to a target site of the subject in need thereof; administering trastuzumab to the target site of the subject in need thereof and further comprising administering a second therapeutic agent to the subject. For example, the second therapeutic agent can be a chemotherapeutic agent. Chemotherapeutic agents can be, but are not limited to, alkylating agents, antimetabolites, anthracyclines, antitumor antibiotics, monoclonal antibodies, platinums, or plant alkaloids.

In some aspects, the trastuzumab is administered in a therapeutically effective amount.

### E. Methods of Increasing Accumulation of an Antibody

Disclosed are methods of increasing accumulation of trastuzumab at a target site of a subject in need thereof comprising applying an alternating electric field, at a frequency for a period of time, to the target site of the subject in need thereof; and administering trastuzumab to the target site of the subject in need thereof, wherein the accumulation of the trastuzumab increases at the target site compared to administering the trastuzumab at the target site without applying an alternating electric field.

In some aspects, an increase of accumulation of trastuzumab is any amount higher than the amount of trastuzumab at the target site without applying an alternating electric field. In some aspects, an increase of accumulation of trastuzumab is an amount one fold, two fold, three fold, or more than the amount of trastuzumab at the target site without applying an alternating electric field.

In some aspects, the target site comprises a cell. For example, the cell can be a cancer cell. In some aspects, a cancer cell can be, but is not limited to, a breast or stomach cancer cell or any Her2 positive cell.

In some aspects, the alternating electric field is applied before, after, or simultaneously with administering the trastuzumab. In some aspects, the trastuzumab is administered prior to, simultaneous with or after applying the alternating electric field. Applying the alternating electric field before administering the trastuzumab can include seconds, minutes, or hours before administering the trastuzumab. Applying the alternating electric field after administering the trastuzumab can include seconds, minutes, hours, or days after administering the antibody. Applying the alternating electric field simultaneously with administering the trastuzumab can include seconds or minutes before or after administering the trastuzumab. In some aspects, simultaneously applying the alternating electric field and the trastuzumab can include administering the trastuzumab while the alternating electric field is being applied. In some aspects, the step of applying the alternating electric field begins at least one hour before the given time.

In some aspects, the trastuzumab comprises a detectable agent. As used herein, a detectable agent, or label, is any molecule that can be associated with trastuzumab, directly or indirectly, and which results in a measurable, detectable signal, either directly or indirectly. Many such labels for conjugating or coupling to an antibody are known to those of skill in the art. Examples of detection agents can be, but are not limited to, radioactive isotopes, fluorescent molecules (fluorophore), phosphorescent molecules, enzymes, antibodies, and ligands.

In some instances, a label can be, but is not limited to, an isotope marker, colorimetric biosensors, or fluorescent labels. For example, fluorescent markers can be, but are not limited to, green fluorescent protein (GFP) or rhodamine fluorescent protein (RFP). Other labels can include biotin, streptavidin, horseradish peroxidase, or luciferase. Other examples of suitable fluorescent labels include, but are not limited to, fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, OR and Research Organics, Cleveland, Ohio.

In some aspects, the frequency of the alternating electric field is any of those described herein. In some aspects, the frequency of the alternating electric field is between 100 and 500 kHz. For example, in some aspects, the frequency of the alternating electric field is 150 kHz.

In some aspects, the alternating electric field has a field strength of any of those described herein. In some aspects, the alternating electric field has a field strength of between 0.5 and 4 V/cm RMS. For example, in some aspects, the alternating electric field has a field strength of 0.9 V/cm RMS.

In some aspects, the trastuzumab is administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g. computed tomography-guided, during surgery or biopsy) or via inhalation. In some aspects, the trastuzumab is administered in a pharmaceutical composition. For example, the pharmaceutical composition can be any of those described herein.

In some aspects, the subject in need thereof has cancer. In some aspects, the cancer can be breast or stomach cancer. For example, the cancer can be a HER2 positive cancer.

In some aspects, one or more of pAKT, pERK, and pHER2 expression is reduced as a result of the increased accumulation of trastuzumab at the target site.

Disclosed are methods of increasing accumulation of trastuzumab at a target site of a subject in need thereof comprising applying an alternating electric field, at a frequency for a period of time, to a target site of the subject in need thereof; administering trastuzumab to the target site of the subject in need thereof, wherein the accumulation of the trastuzumab increases at the target site compared to administering the trastuzumab at the target site without applying an alternating electric field; and further comprising administering a second therapeutic agent to the subject. For example, the second therapeutic agent can be a chemotherapeutic agent. Chemotherapeutic agents can be, but are not limited to, alkylating agents, antimetabolites, anthracyclines, antitumor antibiotics, monoclonal antibodies, platinums, or plant alkaloids.

In some aspects, the trastuzumab is administered in a therapeutically effective amount.

### F. Methods of Inhibiting Cancer Cell Proliferation

Disclosed are methods of inhibiting cancer cell proliferation at a target site of a subject in need thereof comprising applying an alternating electric field, at a frequency for a period of time, to the target site of the subject in need thereof; and administering trastuzumab to the target site of the subject in need thereof, wherein cancer cell proliferation is inhibited.

In some aspects, the increase in the inhibition of cancer cell proliferation is compared to cancer cell proliferation of a target site when alternating electric fields are administered alone, where the trastuzumab is administered alone, or wherein no treatment is administered to the subject. In some aspects, cancer cell proliferation is inhibited compared to administering trastuzumab in the absence of applying an alternating electric field. In some aspects, there is an increase in the inhibition of cancer cell proliferation. For example, an increase in the inhibition of cancer cell proliferation is compared to administering the trastuzumab at the target site without applying an alternating electric field. In some aspects, an increase in the inhibition of cancer cell proliferation is any amount higher than the amount of inhibition of cancer cell proliferation at the target site without applying an alternating electric field. In some aspects, an inhibition of cancer cell proliferation is an amount one fold, two fold, three fold, or more than the amount of inhibition of cancer cell proliferation without applying an alternating electric field.

In some aspects, the target site comprises a cell. For example, the cell can be a cancer cell. In some aspects, a cancer cell can be, but is not limited to, a breast or stomach cancer cell. For example, in some aspects, target site is a cancer cell.

In some aspects, the alternating electric field is applied before, after, or simultaneously with administering the trastuzumab. In some aspects, the trastuzumab is administered prior to, simultaneous with or after applying the alternating electric field. Applying the alternating electric field before administering the trastuzumab can include seconds, minutes, or hours before administering the trastuzumab. Applying the alternating electric field after administering the trastuzumab can include seconds, minutes, hours, or days after administering the trastuzumab. Applying the alternating electric field simultaneously with administering the trastuzumab can include seconds or minutes before or after administering the trastuzumab. In some aspects, simultaneously applying the alternating electric field and the antibody can include administering the trastuzumab while the alternating electric field is being applied. In some aspects, the step of applying the alternating electric field begins at least one hour before the given time.

In some aspects, the trastuzumab comprises a detectable agent. As used herein, a detectable agent, or label, is any molecule that can be associated with trastuzumab, directly or indirectly, and which results in a measurable, detectable signal, either directly or indirectly. Many such labels for conjugating or coupling to an antibody are known to those of skill in the art. Examples of detection agents can be, but are not limited to, radioactive isotopes, fluorescent molecules (fluorophore), phosphorescent molecules, enzymes, antibodies, and ligands.

In some instances, a label can be, but is not limited to, an isotope marker, colorimetric biosensors, or fluorescent labels. For example, fluorescent markers can be, but are not limited to, green fluorescent protein (GFP) or rhodamine fluorescent protein (RFP). Other labels can include biotin, streptavidin, horseradish peroxidase, or luciferase. Other examples of suitable fluorescent labels include, but are not limited to, fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, OR and Research Organics, Cleveland, Ohio.

In some aspects, the frequency of the alternating electric field is any of those described herein. In some aspects, the frequency of the alternating electric field is between 100 and 500 kHz. For example, in some aspects, the frequency of the alternating electric field is 150 kHz.

In some aspects, the alternating electric field has a field strength of any of those described herein. In some aspects, the alternating electric field has a field strength of between 0.5 and 4 V/cm RMS. For example, in some aspects, the alternating electric field has a field strength of 0.9 V/cm RMS.

In some aspects, the trastuzumab is administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g. computed tomography-guided, during surgery or biopsy) or via inhalation. In some aspects, the trastuzumab is administered in a pharmaceutical composition. For example, the pharmaceutical composition can be any of those described herein.

In some aspects, the subject in need thereof has cancer. In some aspects, the cancer can be breast or stomach cancer. For example, the cancer can be a HER2 positive cancer.

In some aspects, one or more of pAKT, pERK, and pHER2 expression is reduced as a result of the increased accumulation of trastuzumab at the target site.

Disclosed are methods of inhibiting cancer cell proliferation at a target site of a subject in need thereof comprising applying an alternating electric field, at a frequency for a period of time, to a target site of the subject in need thereof; administering trastuzumab to the target site of the subject in need thereof, wherein cancer cell proliferation is inhibited; and further comprising administering a second therapeutic agent to the subject. For example, the second therapeutic agent can be a chemotherapeutic agent. Chemotherapeutic agents can be, but are not limited to, alkylating agents, antimetabolites, anthracyclines, antitumor antibiotics, monoclonal antibodies, platinums, or plant alkaloids.

In some aspects, the trastuzumab is administered in a therapeutically effective amount.

### G. Methods of Increasing Apoptosis of Cancer Cells

Disclosed are methods of increasing apoptosis of cancer cells at a target site of a subject in need thereof comprising applying an alternating electric field, at a frequency for a period of time, to the target site of the subject in need thereof; and administering trastuzumab to the target site of the subject in need thereof, wherein apoptosis of cancer cells is increased.

In some aspects, the increased apoptosis of the cancer cells is compared to cell apoptosis of a target site when alternating electric fields are administered alone, where the trastuzumab is administered alone, or wherein no treatment is administered to the subject.

In some aspects, increased apoptosis of the cancer cells is inhibited compared to administering trastuzumab in the absence of applying an alternating electric field. In some aspects, there is an increase in apoptosis of the cancer cells. For example, an increase in apoptosis of the cancer cells is compared to administering the trastuzumab at the target site without applying an alternating electric field. In some aspects, an increase in apoptosis of the cancer cells is any amount higher than the amount of increased apoptosis of the cancer cells at the target site without applying an alternating electric field. In some aspects, an increase in apoptosis of the cancer cells is an amount one fold, two fold, three fold, or more than the amount of increased apoptosis of the cancer cells without applying an alternating electric field.

In some aspects, the target site comprises a cell. For example, the cell can be a cancer cell. In some aspects, a cancer cell can be a breast or stomach cancer cell. For example, in some aspects, target site is a cancer cell.

In some aspects, the alternating electric field is applied before, after, or simultaneously with administering the trastuzumab. In some aspects, the trastuzumab is administered prior to, simultaneous with or after applying the alternating electric field. Applying the alternating electric field before administering the trastuzumab can include seconds, minutes, or hours before administering the trastuzumab. Applying the alternating electric field after administering the trastuzumab can include seconds, minutes, hours, or days after administering the trastuzumab. Applying the alternating electric field simultaneously with administering the trastuzumab can include seconds or minutes before or after administering the trastuzumab. In some aspects, simultaneously applying the alternating electric field and the antibody can include administering the trastuzumab while the alternating electric field is being applied. In some aspects, the step of applying the alternating electric field begins at least one hour before the given time.

In some aspects, the trastuzumab comprises a detectable agent. As used herein, a detectable agent, or label, is any molecule that can be associated with trastuzumab, directly or indirectly, and which results in a measurable, detectable signal, either directly or indirectly. Many such labels for conjugating or coupling to an antibody are known to those of skill in the art. Examples of detection agents can be, but are not limited to, radioactive isotopes, fluorescent molecules (fluorophore), phosphorescent molecules, enzymes, antibodies, and ligands.

In some instances, a label can be, but is not limited to, an isotope marker, colorimetric biosensors, or fluorescent labels. For example, fluorescent markers can be, but are not limited to, green fluorescent protein (GFP) or rhodamine fluorescent protein (RFP). Other labels can include biotin, streptavidin, horseradish peroxidase, or luciferase. Other examples of suitable fluorescent labels include, but are not limited to, fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, OR and Research Organics, Cleveland, Ohio.

In some aspects, the frequency of the alternating electric field is any of those described herein. In some aspects, the frequency of the alternating electric field is between 100 and 500 kHz. For example, in some aspects, the frequency of the alternating electric field is 150 kHz.

In some aspects, the alternating electric field has a field strength of any of those described herein. In some aspects, the alternating electric field has a field strength of between 0.5 and 4 V/cm RMS. For example, in some aspects, the alternating electric field has a field strength of 0.9 V/cm RMS.

In some aspects, the trastuzumab is administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g. computed tomography-guided, during surgery or biopsy) or via inhalation. In some aspects, the trastuzumab is administered in a pharmaceutical composition. For example, the pharmaceutical composition can be any of those described herein.

In some aspects, the subject in need thereof has a cancer selected from breast or stomach cancer. For example, the cancer can be a HER2 positive cancer.

In some aspects, one or more of pAKT, pERK, and pHER2 expression is reduced as a result of the increased accumulation of trastuzumab at the target site.

Disclosed are methods of increasing apoptosis of cancer cells at a target site of a subject in need thereof comprising applying an alternating electric field, at a frequency for a period of time, to a target site of the subject in need thereof; administering trastuzumab to the target site of the subject in need thereof, wherein cancer cell apoptoses is increased; and further comprising administering a second therapeutic agent to the subject. For example, the second therapeutic agent can be a chemotherapeutic agent. Chemotherapeutic agents can be, but are not limited to, alkylating agents, antimetabolites, anthracyclines, antitumor antibiotics, monoclonal antibodies, platinums, or plant alkaloids.

In some aspects, the trastuzumab is administered in a therapeutically effective amount.

### H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising an antibody and one or more materials for delivering alternating electric fields, such as the Optune system.

### EXAMPLES

### A. Example 1. Tumor Treating Fields Can Effectively Overcome Trastuzumab Resistant Breast Cancer Multiplication

The Human Epidermal Growth Receptor 2, or the HER 2, is one of the highest expressed negative receptor that constitutes approximately 15-20% of malevolent breast cancerous tumors among women. The prevalence of HER2 has untimely and unfavorable consequences on breast cancer, and its underlying carcinomous cell processes, structures, and growth. Trastuzumab, a humanized antibody that is rooted in relatively recent foundations, has been found operational in its construction of treatments against HER2-positive breast cancer. This drug is combined with radiotherapy or chemotherapy to deregulate HER2 genes in the body. However, patients who suffer from evolved tumors in advanced stages of cancer exhibit a good amount of tolerance towards singularly used Trastuzumab treatment. Inversely, the factorization of Tumor Testing Fields (TTFields or TTFs) into cancer therapy revives the functions of a Trastuzumab treatment plan, by sensitizing the HER2 genes to the drug. In turn, this facilitates Trastuzumab to continue limiting cancerous cell multiplication and toxicity levels within the treatment. The research evaluates the aspects and effects of this pairing, both in vivo and in vitro through BT474 cells. The TTFields conduct an electromagnetic boundary, which generates sine-wave radiations to manipulate the HER2 gene structure. The methods followed in this example also examine the gene cell cultures and their viability through solutions like Tryptophan blue, or the Crystal violet which may or may not deliver certain testmants to the experiment. The Western Blot Test and the IHC confirm the presence of antibodies and negative receptors in the BT474 cells. These procedures contribute to the formulation of a treatment plan that overcomes the trastuzumab-resistant nature of the tumor, which is essentially the aim of the research. This example shows that a healthy combination of TTFs with Trastuzumab can inhibit the dimerlization and the expression of dangerous gene structures.

1. Materials and Methods
i. Experimental Setup of the Electric Fields

TTFs were generated through a pair of insulated wires attached to a functional generator and amplifier of high voltage, generating sine-wave signals in the range of 0-800 V [17]. It resulted in an applied electric field intensity of 0.9 V/cm, while the frequency was 150 kHz. The field intensity was kept at 0.9 V/cm because of its use in clinical settings. Next, the cells were plated in 100-mm dishes and incubated at 37° C. under humidity as well as 5% CO2 atmosphere, for the purpose of irradiation treatment, until 70-80% confluency was achieved.
ii. Cell Culture

BT-474 and JIMT-1 (ACC-589) ductal carcinoma cells was obtained from the American Type Tissue Culture Collection. BT-474 and JIMT-1 cells were maintained in DMEM-F12 supplemented with 10% heat-inactivated fetal bovine serum (FBS), 2 mmol/L-glutamine, and 1% penicillin G-streptomycin. Cells were maintained at a temperature of 37° C. with 5% CO2.
iii. Cell Viability Assay

Cell viability was determined by trypan blue exclusion assay. An equal volume of trypan blue reagent was added to a cell suspension, and the percentage of viable cells was evaluated by microscopy. Assays were performed in triplicate.
iv. Colony Formation Assay (CFA)

The cells were subjected to the TTFields 6 hours after trastuzumab exposure at a final concentration of 5 μmol/L, after which cells were incubated for 48 hours. After 14-20 days, colonies were stained with 0.4% Crystal Violet (Sigma, St. Louis, MO, USA). The plating efficiency (PE) indicates the percentage of seeded cells of a particular cell line that formed colonies under specific culture conditions. The survival fraction, expressed as a function of irradiation, was calculated as follows: survival fraction=colonies counted/(cells seeded×PE/100).
v. Western Blot Analysis

Total proteins were extracted from OS cells using RIPA buffer (50 mM Tris-Cl, pH 7.4; 1% NP-40; 150 mM NaCl, and 1 mM EDTA), complemented with protease inhibitors (1 mM PMSF, 1 μg/ml aprotinin, 1 μg/ml leupeptin, and 1 mM Na3VO4), and the Bradford method was utilized for quantification. SDS/polyacrylamide gel electrophoresis was used to separate the protein samples (30 μg), which were then transferred to a nitrocellulose membrane. Followed by blocking the non-specific antibody binding sites, the membrane was incubated at 4° C. overnight with mouse monoclonal antibodies. After incubation at 37° C. for 1 hour with peroxidase-conjugated secondary antibodies, the protein bands were visualized by enhanced chemiluminescence reagent (GE Healthcare Biosciences, Pittsburgh, PA, USA) and the Amersham Imager 680 (GE Healthcare Biosciences) was used for detection.
vi. Immunohistochemistry

For the purpose of immunohistochemical assessment, breast segments embedded with paraffin and 4-μm in thickness were mounted on coated glass slides to study the proteins being investigated, followed by the blocking of endogenous peroxidases, retrieval of antigen, and nonspecific protein binding. Then, first the primary antibodies (purchased from Cell Signaling Technology [Danvers, MA, USA]) were used to incubate the slide sections and then horseradish peroxidase-conjugated was used on secondary antibodies. All slides were developed using 3,3′-diaminobenzidine and were counterstained with hematoxylin, following which blind analysis was performed.
vii. Conjugation of Alexa Fluor 488 to Trastuzumab

A solution of Alexa 488-NHS Ester (Invitrogen, Waltham, MA, USA) was prepared using DMSO. It contained 1% acetic acid was dissolved in 500 μL TRZ (10 mg/mL) in 1 M sodium bicarbonate solution with pH of 8.4. The obtained reaction was incubated at room temperature for 1 hour after which it was purified through size exclusion PD-10 column (GE Healthcare Bio-Sciences AB, Uppsala, Sweden) and connected with an ultra-violet/visible detector, which was set at 517-nm maximum wavelength. An aliquot (100 μg/μL, PBS, pH 7.2) of unconjugated was measured along with conjugated Alexa 488-TRZ using Nano Drop Spectrophotometer (ThermoFisher Scientific, Waltham, MA, USA). Using a high-performance liquid chromatography profile, the Alexa 488 molecules surrounding TRZ were estimated through a comparison with the peak intensity between conjugated Alexa 488-TRZ and Alexa 488 free eluted solution.
viii. In Vivo Antibody Penetration Studies

BT-474 cells (5×106) were administered through subcutaneous injection into male BALB/nude mice (n=6 per group). Using a digital caliper, the tumor size was measured and for volume calculation, the following formula was applied: width 2×length×0.5. The protocol by Institutional Animal Care and Use Committee (IACUC) (number KIRAMS 2018-0016; date of approval: 15 May 2018) of Korea Institute of Radiological and Medical Sciences (KIRAMS) was followed for all experiments related to mice (5-6 weeks old; weighing 18-20 grams).

When the tumor size reached ˜200 mm3, 150 μg of Alexa 488-TRZ was intravenously injected, and TTFied (0.9 V/cm) was exposed for a period of 5 days, following which the mice were exsanguinated by cardiac puncture and were dissected. The isolated tumors were fixed immediately with paraformaldehyde (4%) and kept overnight at 4° C. Thereupon, the tumor tissues were embedded in the optimum cutting temperature (OCT) compound and frozen until further use at −70° C. The frozen tissue samples were divided into 8 pm-thick portions with a Leica CM 1850 cryostat (Leica microsystems, USA), rehydrated using PBS, stained with DAPI, and were observed under a fluorescent microscope (In cell analyzer 2200, GE Healthcare, USA). The TUNEL-positive cells were stained with Click-iT® TUNEL Alexa Fluor® 647 Imaging Assay kit (Invitrogen, Carlsbad, CA, USA). Further, the apoptotic cells were calculated by TUNEL assay.
ix. Histological Image Acquisition

Since the In cell analyzer is custom-built with a fluorescent microscope and mosaic stitching software (In cell developer toolbox, GE Healthcare, USA), fluorescent images were obtained with 10× objective lenses using three independent channels. Further, DAPI was used for nuclei (shown in blue), Alexa 488-TRZ (green), and Rhodamine lectin to detect blood vessel (red) images. Offset was determined by Autofocus.
x. Image Analysis of Alexa 488 Trastuzumab Accumulation in Tumor

An in-house program written in MATLAB (Math Works, Natick, MA, USA), ZEN (blue), and MIPAV (NIH, USA) was utilized for image analysis. Individual channels were exported in a TIFF file from ZEN (blue). The derived graph, which depicted line intensity vs line distance, was read using the in-house MATLAB program and MIPAV application (National Institutes of Health, Bethesda, MD, USA). The TRZ signal intensity was noted as 60 μm from the tumor vessel. Line profiling was used to plot the TRZ penetration from the tumor vessel by measuring for each tumor the intensity in various ROI from the vessel from peripheral and central regions. Additionally, the area under the curve was calculated from 10 sections in three tumors for each one in two groups.
xi. Statistical Analysis

Statistical significance was determined using Student's t-test. Differences were considered significant if the P value was less than 0.05, 0.001 or 0.001. (*p<0.05; **P<0.01; ***P<0.001).

2. Results
i. Treatment with TTF Sensitizes Trastuzumab-Resistant BT474 Cell Line in Vitro

To ascertain TTF's ability to control trastuzumab resistance, two HER2-positive (Jimt1 and BT474) cell lines were treated for 48 hours with 1-100 μg (4-fold dilution).

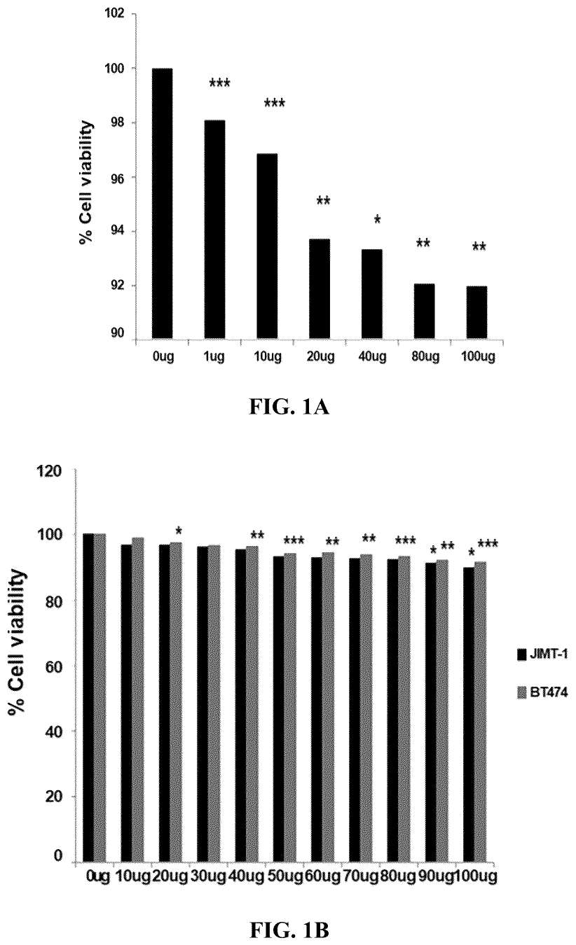

The acquired data indicated that cells display dose-dependent sensitivity to trastuzumab (FIG. 1A). Moreover, treatment with trastuzumab inhibited cell growth significantly in the trastuzumab-resistant cells (FIG. 1B) and both cancer cell lines had decreased cell viability, depending on the various dosage, with approximately 10% viability inhibition observed at 100 μg.
ii. Inhibited Cell Proliferation in Trastuzumab-Resistant HER2-Positive Human Breast Cancer Cell in Vitro After TTF Treatment

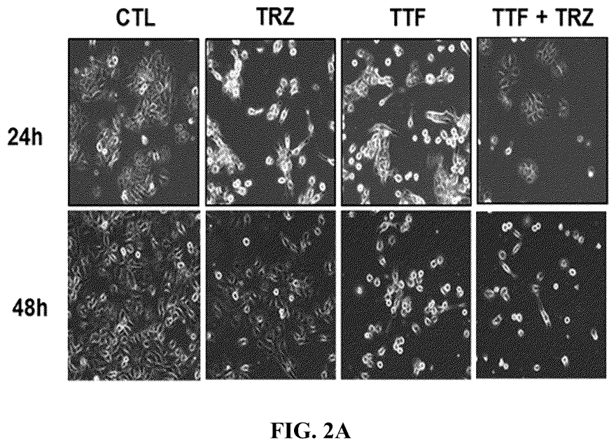

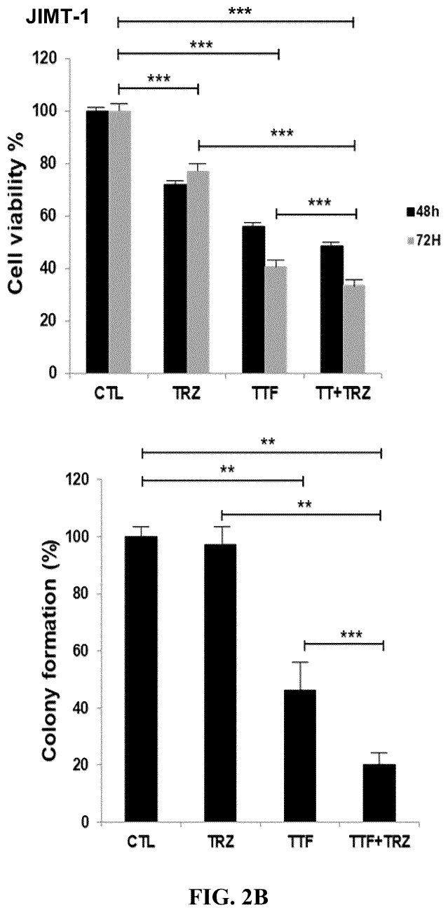

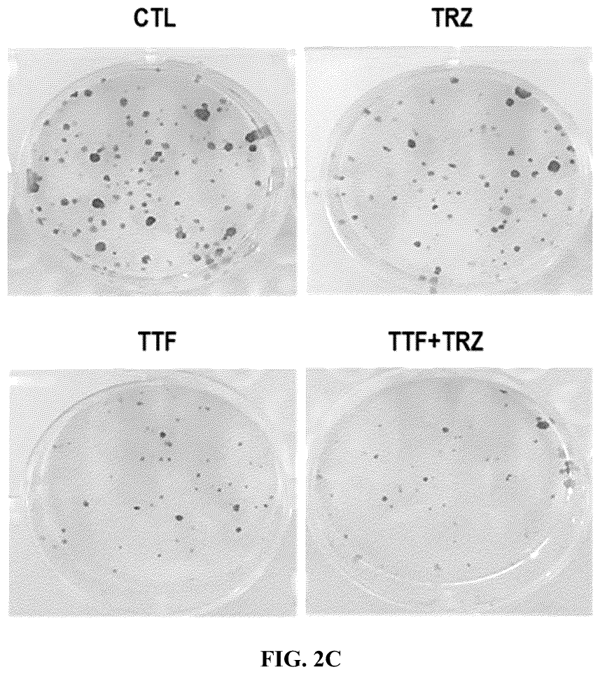

Next, Jimt1 cells was treated with fixed dose of trastuzumab to evaluate its effects on breast cancer cells in cell morphology and 3-(4,5-dimethylthiazol-2-yl)-2,5-diphenyltetrazolium bromide (MTT) assays and colony forming assay (FIG. 2A-2C). Jimt1 cells were treated with TTFs (0.9 V/cm), TRZ, or a combination of the two and the changes related to morphology were observed using a phase-contrast microscope in combined treatment group. Also, the cell growth was noted to be significantly inhibited after a 48-hour treatment at 100 μg trastuzumab. These data show that TTF increased trastuzumab sensitivity in trastuzumab-resistant HER2-positive human breast cancer cell.
iii. Trastuzumab Promotes TTFields Sensitivity in Vivo

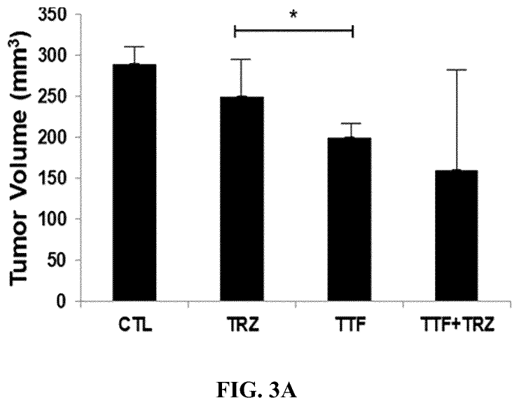

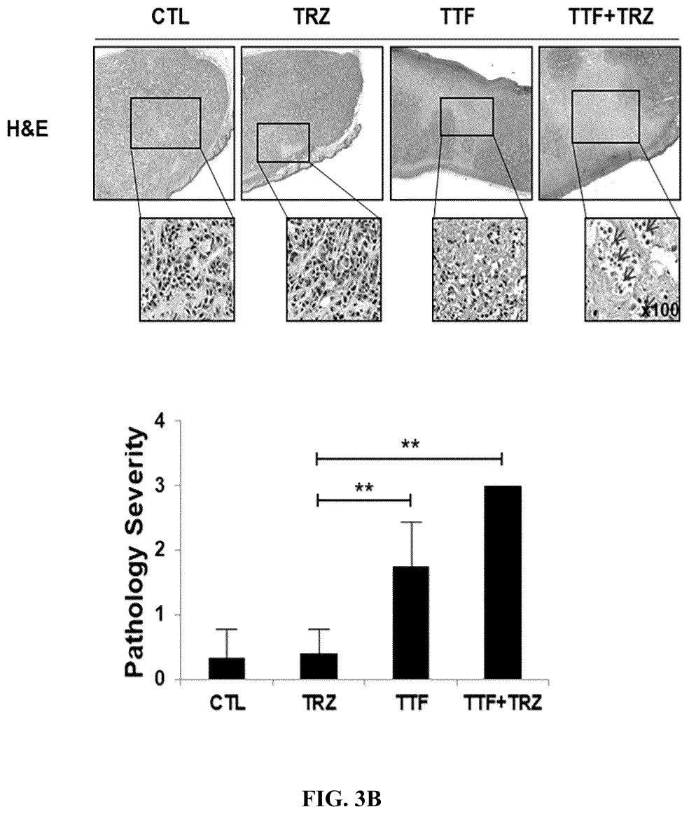

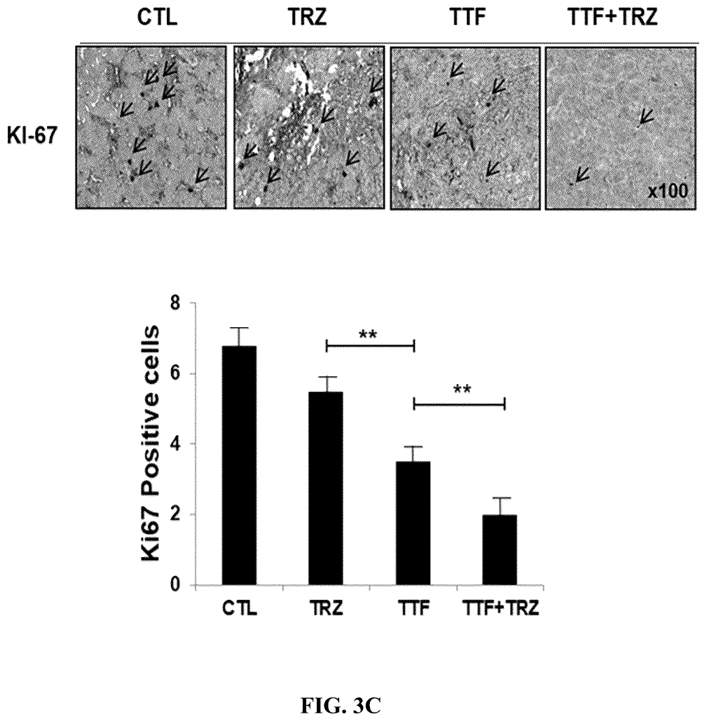

To study TTFs and trastuzumab's combined effect on growth of breast cancer in vivo, a subcutaneous HER2-positive breast cancer model that was achieved by injecting mice with human BT474 cells was utilized. As shown in FIG. 3A, the xenografts treated with TTF and trastuzumab combined displayed growth reduction, as compared to the control group and the groups that received either of the treatments. Thus, mono-treated groups' tumors were markedly larger than those in the combinatorial treatment group. At the same time, the volume of the tumor was reduced in combinatorial treatment mice, compared to the volume in mice that received either of the treatments (FIG. 3A).

Xenografts of mice that received either of the treatments depicted stronger proliferation marker Ki-67 staining than that in combinatorial-treated mice (FIG. 3B-3C). Together, the data show that TTF combined with trastuzumab slows down breast cancer growth in vivo.
iv. Treatment with TTF Plus TRZ Inhibits P-HER2 Expression and Increases Apoptosis Marker Levels in Vitro

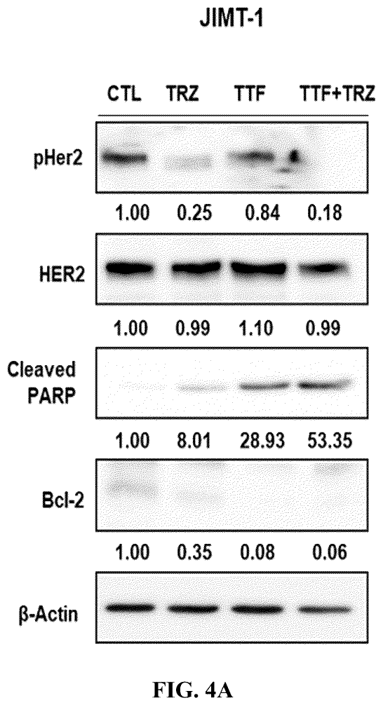

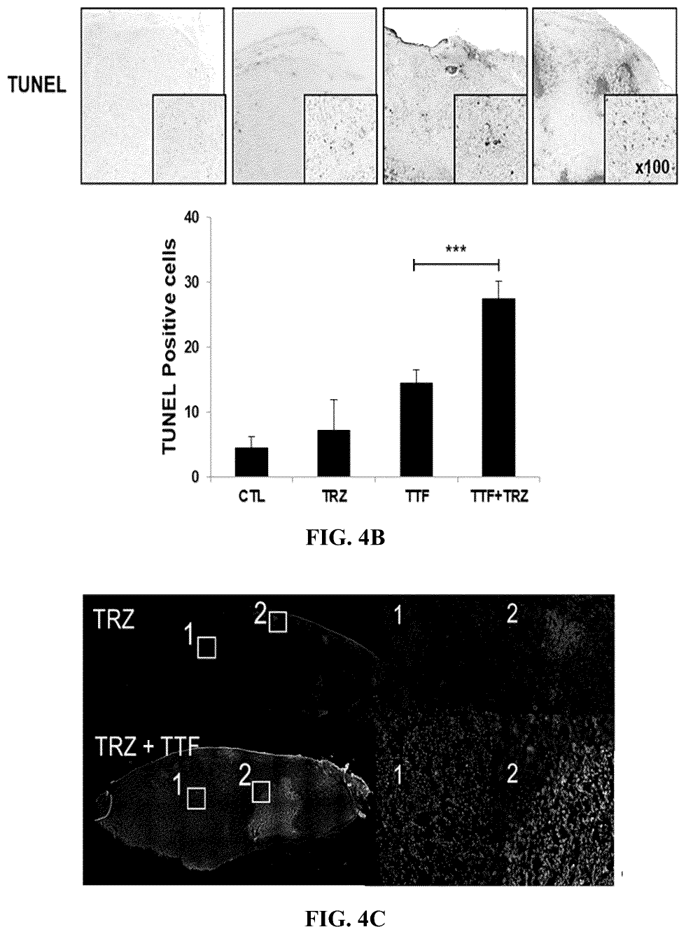

p-HER2′s expression levels and apoptosis marker cleaved PARP as well as anti-apoptosis marker Bcl-2 were studied by the western (or protein immunoblot) blotting technique. The expression levels of p-HER2 and Bcl-2 were downregulated and cleaved PARP was upregulated in Jimt1 cell line following 24 hours treatment with TTF+TRZ (FIG. 4A). To calculate combinatorial therapy's ability to induce apoptosis in vivo, the apoptotic rate was evaluated through a terminal deoxynucleotidyl transferase-mediated dUTP nick-end labeling (TUNEL) assay. Notably, upon combinatorial treatment, the cell death in apoptotic cells went up (FIG. 4B). TTF increased apoptotic region which was calculated by TUNEL assay (FIG. 4C).
v. Treatment with TTF Inhibits P-HER2 and its Downstream Mediators AKT and MAPK Expression Levels in Vivo

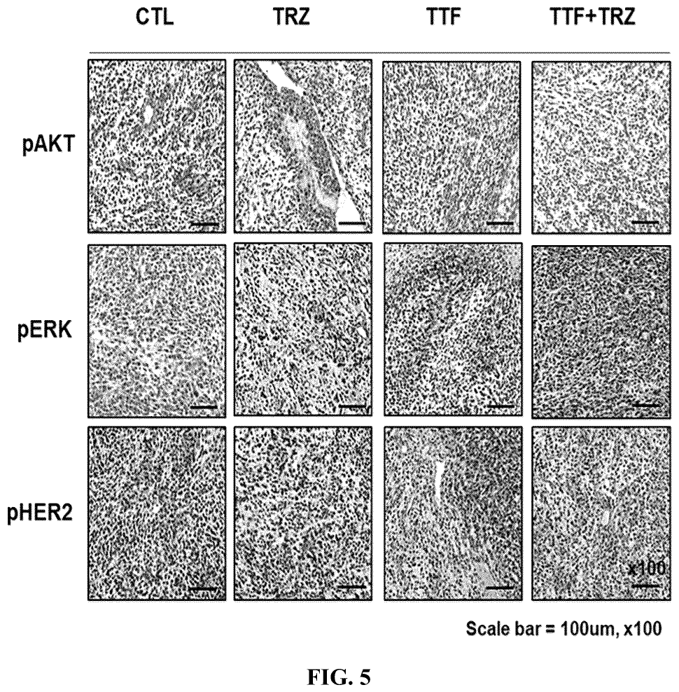

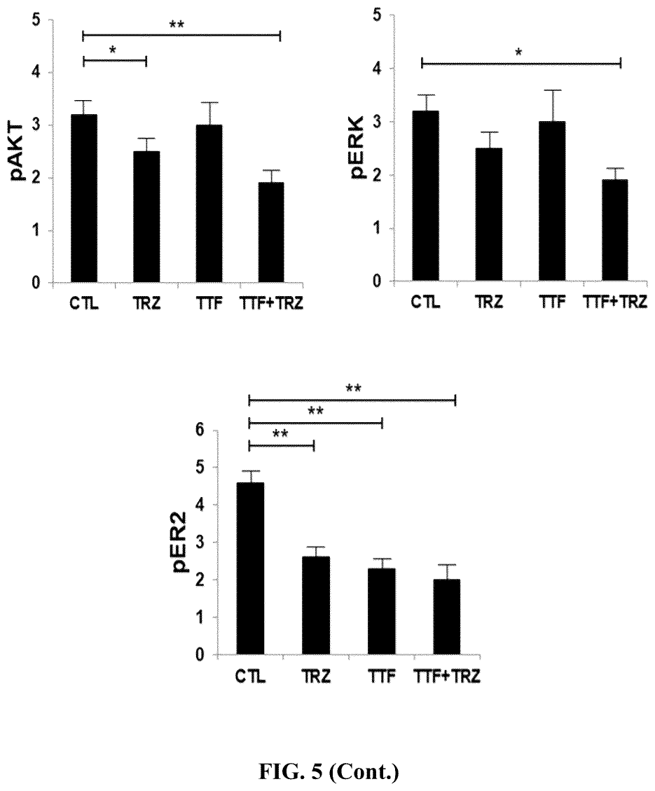

Histopathological studies showed that tumors that were treated with TTF expressed low HER2 levels, as compared with the control group. Moreover, tumors treated using TTF combined with trastuzumab expressed lower levels of pAKT, pERK, and pHER2 in comparison to the control group (FIG. 5).
vi. Treatment with TTF Increases Alexa 488-Trastuzumab Penetration From the Tumor Vessel in Vivo

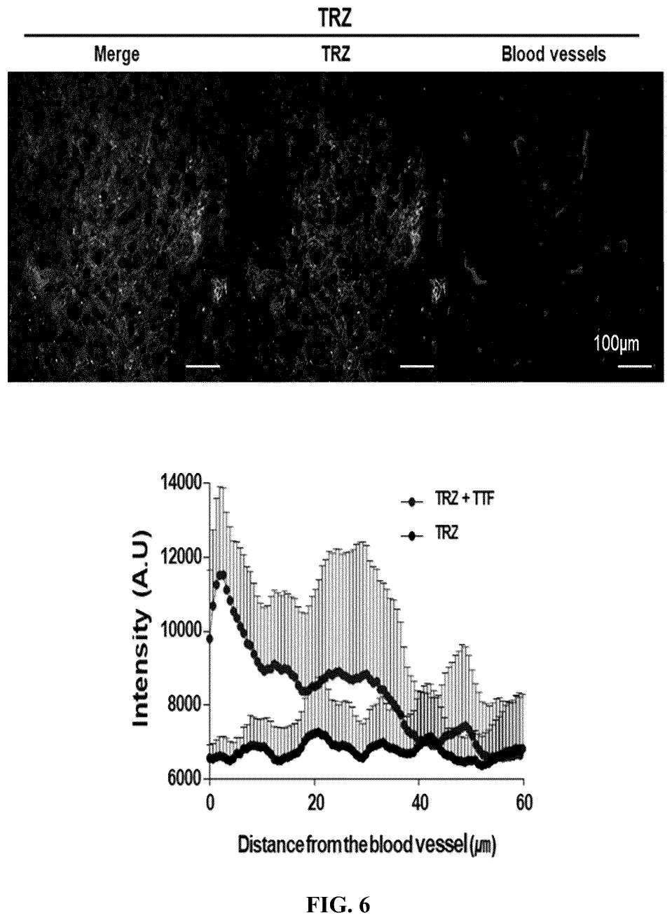

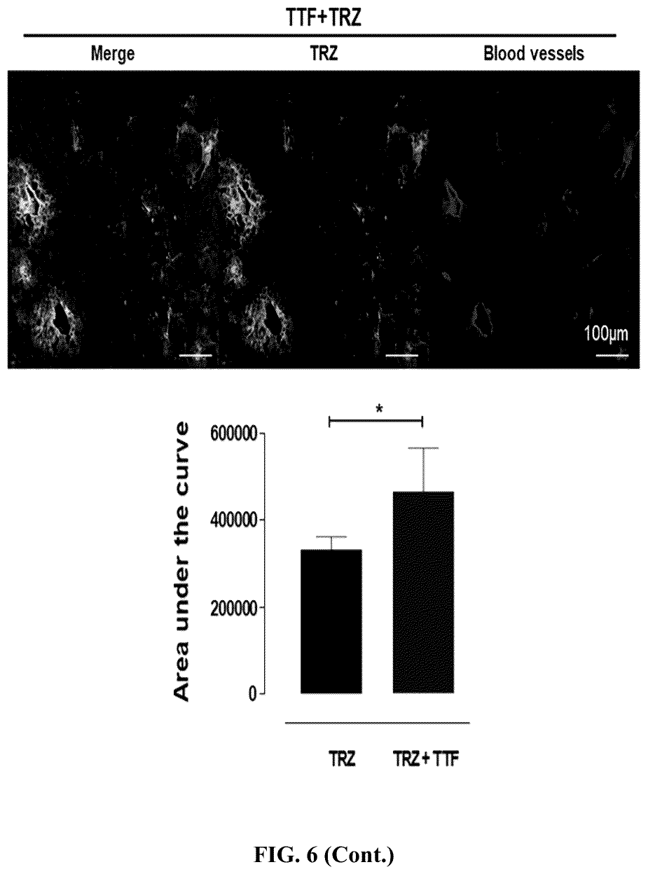

Antibody accumulation was determined by analyzing fluorescence intensity in tumor sections. The result shows that Alexa 488-TRZ (green) accumulation was improved with TTF. The TRZ penetration from the tumor vessel in the entire tumor section was ˜33% greater in TTF combined treated tumors (p=0.0211) (FIG. 6). TTF significantly improved the extravasation of TRZ within 60 μm from the tumor vessel.

3. Discussion

The present study validates the postulation that supporting a generalized trastuzumab drug, which is used to exercise control over life-threatening HER2 positive breast cancerous genes, with Tumor Electrofields Therapy may amplify the inhibition of such trastuzumab resistant cells. The research also aims to identify and unravel the workings of a TTField system on involved trastuzumab-resistant, HER2 positive breast cancer cell lines. By doing so, it brings up the value of TTF's for use in future studies. In September 1998, the FDA approved the use of Trastuzumab and its incorporation in devising the treatment plan of GBM or the Glioblastoma Multiforme, a rare malignant tumor. This need for Trastuzumab application for therapy is issued because of the exaggerated activity of the HER2 tyrosine kinase receptor gene. In response to the proliferation, the rate at which breast cancer progresses into its secondary stages increases alarmingly. Trastuzumab was, thus, predicted to work as a humanized anti-HER2 monoclonal IgG1 antibody [18]. However, this study as well as other instances in the past, evidence clearly that a HER2 gene that is past its primary stages remains indifferent to the influence of trastuzumab. Hence, treatment therapy for breast cancer is at a large disadvantage due to the trastuzumab resistance it faces. Mechanisms that contribute to trastuzumab's failure to operate upon HER2 are intensively studied, and include the following general observations: i) obstacles are faced by trastuzumab in binding to HER2; ii) upregulation of signaling pathways causes HER2 downstream; iii) Trastuzumab's tendency of signaling through alternate pathways, and iv) failure to trigger immune-mediated mechanisms to destroy tumor cells [6].

This study shows that the trastuzumab resistant nature of cancerous genes can be compensated by TTF. This study is first of its' own, that destroys cancerous cells by using the Trastuzumab antibody, and sensitizing it within a well-constructed electromagnetic TTF. This finding was confirmed that the penetration and uptake of Alexa 488-TRZ Trastuzumab was enhanced after TTF treatment. FIG. 4 shows that TTTield increased apoptotic region measured by TUNEL assay. Increased apoptotic region can induce decreased interstitial pressure. Enhanced penetration and uptake of Alexa 488-TRZ could be explained due to decreased interstitial pressure after TTF treatment.

The consequence of these applications is a prohibitive one that suppresses HER2 receptor cells. This study stands in opposition to the present milieu in which oncology regards the combination of chemotherapy with Trastuzumab as the most accurate way of dealing with tumor cells. Despite this promulgation, many patients with HER2-overexpressing breast cancer develop de novo or acquired resistance to such methods. However, the results shown in this study that a TTF integrated solution with trastuzumab can enhance HER2 therapy.

This aim is achieved by analyzing the context-dependent roles that TTF plays in trastuzumab resistant breast cancer cells—the general consensus is a positive outcome.

This aim is supported through previous studies, in which TTF is responsible for GBM arrest, deregulating kinase expression that is dependent on cyclin, inducing caspase-3 activation, as well as spindle formation along with cell death in vitro [20,21]. TTF is also responsible for inhibiting the proliferation and invasion of GBM [3,21]. Further, in vivo and in vitro experiments [16] have agreed at the ideality of TTFields, as it targets cancerous cells specifically, and presents as an attractive alternative to conventional cancer treatment. Numerous in vivo experiments have pointed towards the promising results of TTF's in executing anti-tumor activities. Further, TTF's are also capable of impeding the human lung tumor as demonstrated in the xenograft models and, GBM or GBM patient-derived stem cell tumor-bearing models [22,23].

The disclosed data show a distinct positive outline displayed by the TTF that must be studied to and examined to discuss the therapeutic potential of TTF in combination with trastuzumab in HER2-positive breast cancer. In this research, BT474 cell lines are employed into in vivo and in vitro experiments, and their resultant changes are noted and analyzed. From the observations, it has been shown that the use of a TTField leads to the sensitization of the primary trastuzumab-resistant cell line. Consequently, this facilitates apoptosis by increasing the expression of caspase 3/7 assay. Additionally, TTFs can inhibit phosphorylation of the expression levels of HER2, p-AKT, and p-MAPK in HER2-positive BT474 cells. In turn, this suppresses migration and invasion of cells, and successfully inhibits tumor cell growth in vivo model. By evaluating the pathways, the study also identifies TTF therapy as an alternate solution to patients who are resistant to chemotherapy from antibody like trastuzumab.

In conclusion, the data shown herein shows that TTF can overcome the trastuzumab resistance in vitro and in vivo.

The inventors would like to thank Daegu Catholic University.

Those skilled in the art will recognize, or be able to ascertain using no more than routine experimentation, many equivalents to the specific embodiments of the method and compositions described herein. Such equivalents are intended to be encompassed by the following claims.

1. A method of treating a subject in need thereof comprising:
a) applying an alternating electric field, at a frequency for a period of time, to a target site of the subject in need thereof; and
b) administering a therapeutically effective amount of trastuzumab to the target site of the subject in need thereof.

2. A method of increasing accumulation of trastuzumab at a target site of a subject in need thereof comprising:
a) applying an alternating electric field, at a frequency for a period of time, to the target site of the subject in need thereof; and
b) administering a therapeutically effective amount of trastuzumab to the target site of the subject in need thereof,
wherein the accumulation of the trastuzumab increases at the target site compared to administering the trastuzumab at the target site without applying an alternating electric field.

3-4. (canceled)

5. A method of increasing apoptosis of breast or stomach cancer cells at a target site of a subject in need thereof comprising:
a) applying an alternating electric field, at a frequency for a period of time, to the target site of the subject in need thereof; and
b) administering a therapeutically effective amount of trastuzumab to the target site of the subject in need thereof,
wherein apoptosis of the breast or stomach cancer cells is increased.

6. (canceled)

7. The method of claim 1, wherein the target site comprises one or more breast or stomach cancer cells.

8. The method of claim 1, wherein the alternating electric field is applied before, after, or simultaneously with administering trastuzumab.

9. The method of claim 1, wherein the step of applying the alternating electric field begins at least one hour before the given time.

10. The method of claim 1, wherein the trastuzumab comprises a detectable agent.

11. The method of claim 10, wherein the detectable agent is a fluorophore.

12. The method of claim 1, wherein the frequency of the alternating electric field is between 100 kHz and 1 MHz.

13. The method of claim 1, wherein the frequency of the alternating electric field is between 100 kHz and 500 kHz.

14. The method of claim 1, wherein the alternating electric field has a field strength of between 0.5 and 4 V/cm RMS.

15. The method of claim 1, wherein the alternating electric field has a field strength of at least 0.9 V/cm RMS.

16. The method of claim 1, wherein the trastuzumab is administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g. computed tomography-guided, during surgery or biopsy) or via inhalation.

17. The method of claim 1, wherein the trastuzumab is administered in a pharmaceutical composition.

18. The method of claim 1, wherein the subject in need thereof has a cancer selected from breast or stomach cancer.

19. The method of claim 18, wherein the cancer is breast cancer and the subject has trastuzumab resistance.

20. The method of claim 18, wherein the cancer is a HER2 positive cancer.

21. The method of claim 18, wherein pAKT, pERK, and pHER2 expression is reduced.

22. The method of claim 1, wherein the trastuzumab is administered prior to, simultaneous with or after applying the alternating electric field.

23. The method of claim 1, further comprising administering to a chemotherapeutic agent to the subject.

24. (canceled)
